(19) United States
(12) Patent Application Publication
(10) Pub. No.: US 2016/0082075 A1
(43) Pub. Date: Mar. 24, 2016

(54) Combination Therapy for Subcutaneous Administration of Glycopeptide Antibiotics

(71) Applicants: Pieter MUNTENDAM, (US); SCPHARMACEUTICALS, LLC, Boston, MA (US)

(72) Inventor: Pieter Muntendam, Lexington, MA (US)

(73) Assignee: scPharmaceuticals Inc., Lexington, MA (US)

(21) Appl. No.: 14/779,693

(22) PCT Filed: Mar. 25, 2014
(86) PCT No.: PCT/US2014/031683

§ 371 Date: Sep. 24, 2015

Related U.S. Application Data
(60) Provisional application No. 61/805,659, filed on Mar. 27, 2013.

(51) Int. Cl. A61K 38/14; A61K 31/4741; A61K 31/352

(52) U.S. Cl. CPC A61K 38/14; A61K 31/352; A61K 31/4741

(57) ABSTRACT

A combination drug therapy is disclosed for the treatment of a patient, such as a human, with a glycopeptide antibiotic. The methods can include subcutaneous administration to a patient of a therapeutically effective amount of a glycopeptide antibiotic, for example, vancomycin, and a compound belonging to the cromones class of anti-inflammatory agents such as cromolyn sodium. The present teachings also provide a therapeutic combination and a kit including the therapeutic combination.

### BACKGROUND

The present teachings relate to the administration of glycopeptide antibiotics. More specifically, the present teachings relate to a combination therapy of a glycopeptide antibiotic and a cromone.

Glycopeptide antibiotics such as vancomycin can be used in the prophylaxis and treatment of infections caused by Gram-positive bacteria, including treatment of serious infections caused by organisms susceptible to resistance to penicillins, for example, methicillin-resistant Staphylococcus aureus (“MRSA”). Oral bioavailability of glycopeptide antibiotics is poor and it must be given by intravenous infusions for most infections.

Intravenous administration of vancomycin can cause the release of histamine from circulating cells such as basophils and tissue mast cells. Such action can cause two types of hypersensitivity reactions: red man syndrome and anaphylaxis. Red man syndrome is an infusion-related reaction which typically consists of pruritus, an erythematous rash that involves the face, neck, and upper torso. Other signs and symptoms may also occur. Accordingly, the release of histamine and other mediators of inflammation renders glycopeptide antibiotics unsuitable for subcutaneous administration because of the possible adverse reaction at the injection site that may be followed by a systemic reaction.

Thus, there is a need to improve the convenience and safety for the delivery of glycopeptide antibiotics.

### SUMMARY

The present teachings relate to a combination drug therapy for the treatment of certain bacterial infections with subcutaneous administration of a combination of a therapeutically effective amount of a glycopeptide antibiotic and a member of the cromones class of anti-inflammatory agents (a “cromone”). Without wishing to be bound to any particular theory, it is believed that the cromone can block the cellular reactions, such as the release of histamine and other inflammatory mediators, caused by the glycopeptide antibiotic. Thus, the cromone can inhibit the release of inflammatory mediators thereby reducing the risk of local and systemic reactions. Subcutaneous administration of glycopeptide antibiotics, such as vancomycin, can improve the safety of administration and can reduce the cost of therapy when compared to intravenous administration.

Accordingly, in one aspect, the present teachings can provide methods for improving the safety and tolerance of treatment with a glycopeptide antibiotic by administering the glycopeptide antibiotic with a cromone. In various embodiments, the methods can include treating a bacterial infection or alleviating one or more symptoms thereof in a patient. Such methods can include administering subcutaneously to a patient a therapeutically effective amount of a glycopeptide antibiotic and a cromone.

In some embodiments of the methods, the glycopeptide antibiotic can be a lipoglycopeptide antibiotic. In certain embodiments, the glycopeptide antibiotic is selected from vancomycin, teicoplanin, telavancin, oritavancin, dalbavancin, bleomycin, ramoplanin, decaplanin, and combinations thereof.

In various embodiments of the methods, the cromone is selected from cromolyn, nedocromil, and combinations thereof. In some embodiments, the cromone can be the compound. In certain embodiments, the cromone can be a pharmaceutically acceptable salt, hydrate, or ester thereof. For example, the cromone can be selected from cromolyn sodium, nedocromil sodium, and combinations thereof.

In another aspect, the present teachings include a therapeutic combination, for example, a therapeutic preparation, including a glycopeptide antibiotic and a cromone.

In yet another aspect, the present teachings include a kit, where the kit includes a therapeutic combination that includes a glycopeptide antibiotic and a cromone; and instructions for the use of the therapeutic combination.

The foregoing as well as other features and advantages of the present teachings will be more fully understood from the following description, examples, and claims.

### DETAILED DESCRIPTION

The present teachings can enable the administration of glycopeptide antibiotics, for example, vancomycin, by combining the glycopeptide antibiotic(s) with a cromone such as cromolyn, cromolyn sodium, nedocromil, and nedocromil sodium. Subcutaneous administration can improve the safety of glycopeptide antibiotics as well as reduce the cost of therapy when compared to intravenous administration.

Throughout the application, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited process steps.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present teachings remain operable. Moreover, two or more steps or actions may be conducted simultaneously.

As used herein, “patient” refers to a mammal, such as a human.

As used herein, a “compound” refers to the compound itself and its pharmaceutically acceptable salts, hydrates and esters, unless otherwise understood from the context of the description or expressly limited to one particular form of the compound, i.e., the compound itself, or a pharmaceutically acceptable salt, hydrate or ester thereof.

As used herein, a “cromone” refers to a compound which is a member of the cromones class of anti-inflammatory agents. Examples of a cromone include cromolyn, nedocromil, and combinations thereof. A cromone includes the compound, and pharmaceutically acceptable salts, hydrates and esters thereof, for example, cromolyn sodium and nedocromil sodium.

As used herein, a “glycopeptide antibiotic” refers to a compound which is a member of the glycopeptide antibiotic class of anti-microbial agents. A glycopeptide antibiotic generally is a glycosolated cyclic or polycyclic nonribosomal peptide. A glycopeptide antibiotic can include a glycopeptide antibiotic derivative such as a lipoglycopeptide antibiotic. Examples of glycopeptide antibiotics include vancomycin, teicoplanin, telavancin, oritavancin, dalbavancin, bleomycin, ramoplanin, and decaplanin.

As used herein, “therapeutic combination” refers to a combination of one or more active drug substances, i.e., compounds having a therapeutic utility. Typically, each such compound in the therapeutic combinations of the present teachings can be present in a pharmaceutical composition comprising that compound and a pharmaceutically acceptable carrier. The compounds in a therapeutic combination of the present teachings can be administered simultaneously, together or separately, or separately at different times, as part of a regimen.

The present teachings also provide pharmaceutical compositions that include at least one compound described herein or a therapeutic combination, and one or more pharmaceutically acceptable carriers, excipients, or diluents. Examples of such carriers are well known to those skilled in the art and can be prepared in accordance with acceptable pharmaceutical procedures, such as, for example, those described in Remington: The Science and Practice of Pharmacy, 20th edition, ed. Alfonso R. German), Lippincott Williams & Wilkins, Baltimore, Md. (2000), the entire disclosure of which is incorporated by reference herein for all purposes.

As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the formulation and are biologically acceptable. Supplementary active ingredients can also be incorporated into the pharmaceutical compositions.

Compounds and therapeutic combinations of the present teachings can be useful for treating a pathological condition or disorder in a patient, for example, a human. As used herein, “treating” refers to partially or completely alleviating and/or ameliorating the condition and/or symptoms thereof. The present teachings accordingly include a method of providing to a patient a pharmaceutical composition that includes a compound or therapeutic combination of the present teachings in combination or association with a pharmaceutically acceptable carrier. Compounds and therapeutic combinations of the present teachings can be administered alone or in combination with other therapeutically effective compounds or therapies for the treatment of a pathological condition or disorder.

As used herein, “therapeutically effective” refers to a substance or an amount that elicits a desirable biological activity or effect.

Liquid carriers can be used in preparing solutions, suspensions, and emulsions. A compound described herein can be dissolved or suspended in a pharmaceutically acceptable liquid carrier such as water, an organic solvent, or a mixture of both, or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, and osmo-regulators. Examples of liquid carriers for parenteral administration include water, alcohols (including monohydric alcohols and polyhydric alcohols, e.g., glycols) and their derivatives, and oils (e.g., fractionated coconut oil and arachis oil). For parenteral administration, the carrier can be an oily ester such as ethyl oleate and isopropyl myristate. Sterile liquid carriers are used in sterile liquid form compositions for parenteral administration.

Liquid pharmaceutical compositions, which are sterile solutions or suspensions, can be utilized by, for example, subcutaneous injection.

When administered for the treatment or inhibition of a particular disease state or disorder, it is understood that an effective dosage can vary depending upon many factors such as the particular compound or therapeutic combination utilized, the mode of administration, and severity of the condition being treated, as well as the various physical factors related to the individual being treated. In therapeutic applications, a compound or therapeutic combination of the present teachings can be provided to a patient already suffering from a disease, for example, bacterial infection, in an amount sufficient to cure or at least partially ameliorate the symptoms of the disease and its complications. The dosage to be used in the treatment of a specific individual typically must be subjectively determined by the attending physician. The variables involved include the specific condition and its state as well as the size, age and response pattern of the patient.

The compounds and therapeutic combinations described herein can be administered parenterally. Solutions or suspensions of these active compounds or pharmaceutically acceptable salts, hydrates, or esters thereof can be prepared in water suitably mixed with a surfactant such as hydroxyl-propylcellulose. Dispersions can also be prepared in glycerol, liquid polyethylene glycols, and mixtures thereof in oils. In certain embodiments, a parenteral preparation can include a preservative to inhibit the growth of microorganisms. However, in some embodiments, the parenteral preparation is preservative-free. In particular embodiments, a parenteral preparation can include a buffer as well as other suitable pharmaceutical additives mentioned herein such as solubilizers, emulsifiers, preservatives, sweeteners, flavoring agents, suspending agents, thickening agents, colors, viscosity regulators, stabilizers, and osmo-regulators.

The pharmaceutical forms suitable for injection can include sterile aqueous solutions or dispersions and sterile powders for the extemporaneous preparation of sterile injectable solutions or dispersions. In certain embodiments, the pharmaceutical form is sterile and its viscosity permits it to flow through a syringe. The pharmaceutical form should be stable under the conditions of manufacture and storage, for example, preserved against the contaminating action of microorganisms such as bacteria and fungi, if needed. The carrier can be a solvent or dispersion medium containing liquids such as water, ethanol, polyol (e.g., glycerol, propylene glycol, and liquid polyethylene glycol), suitable mixtures thereof, and vegetable oils.

The following examples are provided to illustrate further and to facilitate the understanding of the present teachings and are not in any way intended to limit the invention.

### Example 1

### Patient with MRSA Skin Infection

A patient with a confirmed MRSA skin infection sensitive to telavancin is discharged from the hospital after 4 days of treatment with intravenous (“iv”) telavancin. The patient is prescribed a combination of telavancin and nedocromil using subcutaneous self administration using a mini-pump for 10 days to complete the course of antimicrobial therapy.

### Example 2

### Patient with Infective Endocarditis with Prosthetic Valve

A patient with infective endocarditis with a prosthetic valve is prescribed an oral regimen of rifampin in combination with a six week course of a combination of vancomycin and cromolyn using subcutaneous self administration using a mini-pump.

### Example 3

### Patient with Osteomyelitis Due to MRSA

A patient with an osteomyelitis of the lower leg following a car accident is prescribed a twelve week course of a combination of vancomycin and cromolyn using subcutaneous self administration using a mini-pump.

The present teachings encompass embodiments in other specific forms without departing from the spirit or essential characteristics thereof. The foregoing embodiments are therefore to be considered in all respects illustrative rather than limiting on the present teachings described herein. Scope of the present invention is thus indicated by the appended claims rather than by the foregoing description, and all changes that come within the meaning and range of equivalency of the claims are intended to be embraced therein.

What is claimed is:

1. A method of treating a bacterial infection or alleviating one or more symptoms thereof in a patient, the method comprising:
administering subcutaneously to a patient a therapeutically effective amount of a glycopeptide antibiotic and a cromone.

2. The method of claim 1 wherein the glycopeptide antibiotic is vancomycin.

3. The method of claim 1 wherein glycopeptide antibiotic is teicoplanin.

4. The method of claim 1 wherein the glycopeptide antibiotic is a lipoglycopeptide.

5. The method of claim 4 wherein the lipoglycopeptide is telavancin.

6. The method of claim 4 wherein the lipoglycopeptide is oritavancin.

7. The method of claim 4 Wherein the lipoglycopeptide is dalbavancin.

8. The method of claim 2 wherein the cromone is cromolyn.

9. The method of claim 2 wherein the cromone is nedocromil.

10. A therapeutic combination comprising a glycopeptide and a cromone.

11. A kit comprising:
a therapeutic combination of claim 10; and
instructions for use of the therapeutic combination.
